(19) United States
(12) Patent Application Publication
(10) Pub. No.: US 2024/0109064 A1
(43) Pub. Date: Apr. 4, 2024

(54) CUTTING SUBASSEMBLY AND CONDITIONING ASSEMBLY FOR AN ENVIRONMENT

(71) Applicant: RAPID MICRO BIOSYSTEMS, INC., Lowell, MA (US)

(72) Inventors: Hunter BLACK, Lowell, MA (US); David LEFORT, Lowell, MA (US); Blair MORAD, Ipswich, MA (US); Victor CHUNG, Melrose, MA (US); Kaitlin BEACH, Shrewsbury, MA (US); Jason MARTIN, Sudbury, MA (US); Frank Owen GRIFFIN, Boston, MA (US); Ethan FRANCO, Dorchester, MA (US); Kayla VOLIS, Dracut, MA (US); Toby ISAACS, Rutland, MA (US); Taylor PARADIS, Chelmsford, MA (US)

(21) Appl. No.: 18/480,853

(22) Filed: Oct. 4, 2023

Related U.S. Application Data
(60) Provisional application No. 63/378,334, filed on Oct. 4, 2022.

(51) Int. Cl. B01L 3/00; C12M 1/32; C12Q 1/04

(52) U.S. Cl. CPC B01L 3/502715; C12M 23/12; C12Q 1/045; B01L 2200/0689; B01L 2200/16; B01L 2300/044; B01L 2300/0672

(57) ABSTRACT

This application relates to improvements in environments for microbial testing. Exemplary embodiments provide a uniquely configured tray containing an environment conditioning material, such as an oxygen scavenger material, for use with a cassette assembly. Furthermore, some embodiments provide a cutting mechanism that allows the tray to be reliably breached, thereby exposing the material inside to the environment. Some embodiments can cut open the material with specially-configured cutter teeth, which can easily be deployed by twisting or pushing two halves of the cassette assembly.

### RELATED APPLICATIONS

This application claims the benefit of U.S. Provisional Patent Application No. 63/378,334, filed Oct. 4, 2022, the entire contents of which are incorporated herein by reference in its entirety.

### BACKGROUND

Testing for microbial contamination often takes place in a sealed environment such as a testing cassette. A sample may be exposed to a growth medium in the environment and any microbial colonies in the sample are allowed to grow for a period of time. After the period of time elapses, the colonies are identified and quantified.

Some microbial tests are performed in an anaerobic (low- or no-oxygen) environment. In order to perform such tests, oxygen must generally be removed from the environment. This can be accomplished by using an oxygen-scavenging material.

Unfortunately, it can be difficult to deploy the scavenging material in the environment successfully. Before the scavenging material is deployed it must be isolated from the air, but must be capable of being exposed to the testing environment after it is assembled (e.g., after the various parts of the cassette are placed together). The packaging for isolating the scavenging material must be sufficiently robust that it will not accidentally expose the material to the air while it is being handled, but at the same time must be capable of being easily and reliably opened while in the sterile environment of the cassette. Furthermore, whatever packaging is used needs to be filled with scavenging material, again in a manner that avoids exposing the scavenging material to the air as much as possible.

### BRIEF SUMMARY

This application relates in some embodiments to improvements in anaerobic (low- or no-oxygen) environments for microbial testing and to conditioning an environment with a material for testing. Exemplary embodiments may provide a uniquely configured tray containing an oxygen-scavenging material or other environmentally conditioning materials for use with a cassette assembly. Furthermore, some embodiments provide a cutting mechanism that allows the tray to be reliably breached, thereby exposing the scavenging material inside to the environment. Some embodiments can cut open the material with specially-configured cutter teeth, which can easily be deployed by twisting or pushing two halves of the cassette assembly.

In one aspect, a cutter for a cassette assembly includes a tray topped with a seal and a lid. The cutter may include an annular planar surface with one or more openings and at least one flexible cutter tooth extending away from an axial top of the planar surface. The flexible cutter tooth may be configured to interact with the lid in order to cause the flexible cutter tooth to flex. A cutting tip on each of the at least one flexible cutter teeth may be sized so that, when engaged with the lid, the cutting tip penetrates the seal on the tray.

The at least one cutter tooth may further be sized so that, when not engaged with the lid, the cutting tip is disposed in one of the openings and does not extend beyond an axial bottom of the cutter.

The at least one cutter tooth may be configured to interface with a ramp on the lid of the cassette assembly to force the cutting tip into the seal.

The cutter may be a ring separate from the lid.

The sealing material may be a foil.

The one or more flexible cutter teeth may include a plurality of teeth arranged substantially around the circumference of the cutter.

The cutter may be configured to penetrate the foil upon a relative twisting motion between the lid and a remainder of the cassette assembly, and/or upon a relative pushing motion between the lid and a remainder of the cassette assembly.

The cutter may be sized and shaped to surround an internal testing environment of the cassette assembly.

The cutter may also include a tooth-free region configured to receive a tool.

An exemplary method may include assembling the above-described cutter into the cassette assembly and seating the lid onto the cassette assembly to cause the cutting tip to penetrate the seal.

Seating the lid may include pushing the lid onto a remainder of the cassette assembly.

The method may also include aligning one or more ramps on the lid with the at least one flexible cutter teeth of the cutter.

The method may also include where twisting the lid over a remainder of the cassette assembly. Twisting the lid may rotate one or more ramps on the lid into the at least one flexible cutter teeth of the cutter.

The method may also include placing the tray into a recess in the cassette assembly.

Assembling the cutter into the cassette assembly may include placing the cutter on top of the tray.

Seating the lid onto the cassette assembly may create an air-tight seal.

The method may also include deploying a tool on a tooth-free region on the cutter.

The method may also include aligning a protrusion on an axial bottom of the cutter with a cut-out portion of the tray.

In one aspect, a tray assembly for a cassette assembly may be provided. The assembly may include a tray configured to receive an oxygen-scavenging material. The tray may be sized and shaped to fit into a recess in a microbial testing cassette. The assembly may further include a seal configured to create an airtight seal on one side of the tray, and one or more fill ports configured to receive the oxygen-scavenging material.

The tray assembly may also include a shelf configured to be disposed inside the tray. A foam insert may be configured to sit on the molded shelf and prevent the material from exiting the tray when the seal is penetrated.

The seal may be a foil or a gas-permeable membrane. The seal may include an adhesive provided around an edge of the seal. The seal may be heat welded in some embodiments.

The tray may be configured to substantially surround an internal testing environment of the cassette assembly. For example, the tray assembly may be “C”-shaped. The one or more fill ports may include at least one fill port provided at each end of the “C” shape.

The tray assembly may also include an air-tight seal provided on the one or more fill ports.

The tray assembly may also include a ring-shaped foil cutter configured to penetrate the seal.

An exemplary method includes providing the tray assembly described above, filling the tray with material through the one or more fill ports, and applying the seal to seal the tray.

The method may also include deploying a shelf inside the tray.

The method may also include deploying a foam insert on top of the shelf, the foam insert configured to prevent the material from exiting the tray when the seal is penetrated.

The method may also include providing an adhesive around an edge of the seal. The seal may be sealed to the tray with the adhesive. In some embodiments, sealing the seal may include applying heat to activate a heat-sensitive adhesive or heat welding a polymer adhesive.

The tray may include a plurality of fill ports and filling the tray may include adding the oxygen scavenging material to each of the fill ports. The one or more fill ports may be sealed.

The method may also include deploying a cutter on top of the tray subassembly. The cutter may be pushed or twisted to cause the cutter to penetrate the seal.

Other technical features may be readily apparent to one skilled in the art from the following figures, descriptions, and claims.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an exemplary cassette assembly in accordance with one embodiment.

FIG. 2 is a close-up of a portion of a cross-sectional view of the cassette assembly in accordance with one embodiment.

FIG. 3 is a close-up of a portion of a cross-sectional view of the cassette assembly in accordance with one embodiment.

FIG. 4 is a perspective view of a foil cutter 106 in accordance with one embodiment.

FIG. 5A is a top view of a foil cutter 106 in accordance with one embodiment.

FIG. 5B is a side view of a foil cutter 106 taken along the line A-A in FIG. 5A in accordance with one embodiment.

FIG. 5C is a side view of a foil cutter 106 taken along the line B-B in FIG. 5A in accordance with one embodiment.

FIG. 5D is a close-up of the detail C (a cutter tooth 402) in accordance with one embodiment.

FIG. 6 is a cutaway side view showing the cutting action of the foil cutter 106 in accordance with exemplary embodiments.

FIG. 7 is an exploded view of an exemplary tray assembly 700 in accordance with one embodiment.

FIG. 8 is a perspective view depicting the assembled tray assembly 700 from the top in accordance with one embodiment.

FIG. 9 is a perspective view depicting the assembled tray assembly 700 from the bottom in accordance with one embodiment.

FIG. 10 is a bottom view of an assembled tray assembly 700 in accordance with one embodiment.

FIG. 11 is a flowchart depicting an exemplary method for creating a limited-oxygen environment in a cassette assembly in accordance with one embodiment.

FIG. 12 shows an example portion of a cutter tooth arrangement having a polymer support arm in accordance with an embodiment.

FIG. 13 shows an illustrative cutter head from a front view in accordance with an embodiment.

FIG. 14 shows an illustrative cutter head with a flat profile in accordance with an embodiment.

FIG. 15 shows an illustrative cutter head with a curved profile in accordance with an embodiment.

FIG. 16 shows an example of how a metal cutter head can be formed by die cutting, punching or photo-etching in accordance with an embodiment.

FIG. 17 shows an illustrative element from the sheet of FIG. 16.

FIG. 18 shows how the element of FIG. 17 may be fit into a foil cutter in accordance with an embodiment.

FIG. 19 depicts an example foil cutter with a ring structure in accordance with an embodiment.

### DETAILED DESCRIPTION

Microbial testing may be performed in an environment which may be created (for example) in a cassette assembly. This application relates in some embodiments to techniques for deploying a material in such a cassette assembly in a reliable and easy-to-use manner. Exemplary embodiments may provide a uniquely configured tray containing an oxygen-scavenging material or other environmental conditioning chemistry for use with a cassette assembly. Further embodiments provide a cutting mechanism that allows the tray to be reliably breached, thereby exposing the material inside to the environment. Some embodiments can cut open the material with specially-configured cutter teeth, which can easily be deployed by twisting or pushing two halves of the cassette assembly. As a result, the cassette assembly can be closed, and as part of the action used to close the assembly, the tray can be breached. Where the material is a scavenging material, this causes the scavenging material to be exposed to the environment, creating an anaerobic condition in the cassette assembly.

Although embodiments described below apply a particular configuration suitable for use with the depicted cassette assembly, one of ordinary skill in the art will recognize that this specific configuration is provided for illustration purposes. The described cutter and/or tray may take on different shapes or sizes depending on the environment in which they are to be deployed. The cutter and/or tray may be used separately to obtain the advantages noted, or together to achieve additional synergies. Furthermore, the cutter and tray need not only be deployed in the context of microbial testing but may be broadly applicable in any circumstance in which a material can be maintained in the tray and deployed using the cutter. The tray may be used in applications other than creating an anaerobic environment but more generally may be used for environmental conditioning that uses desiccants, gases, and/or chemicals which react to create environment to meet particular needs of organisms that are to be detected.

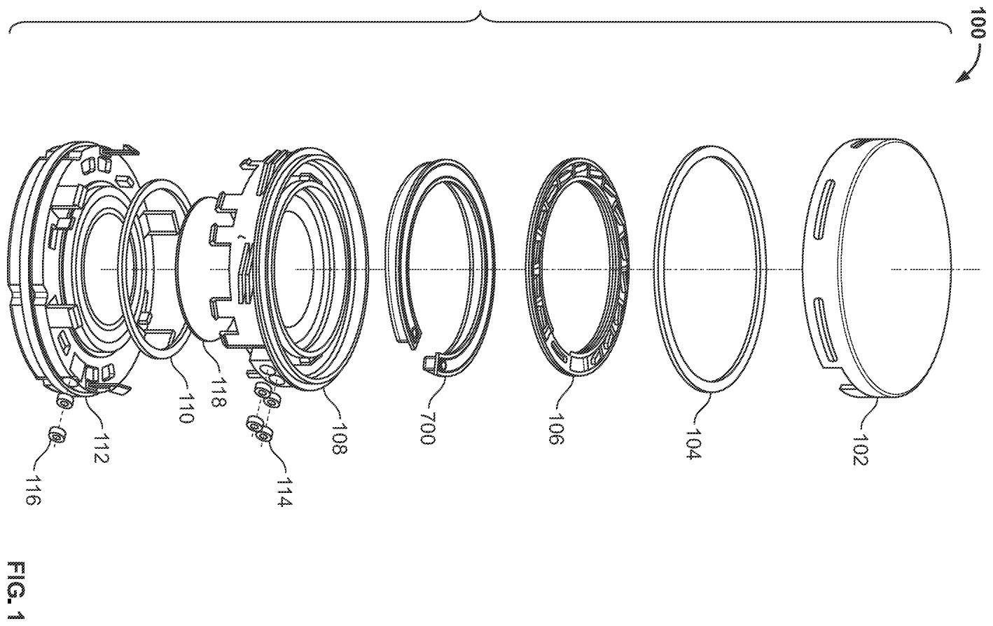

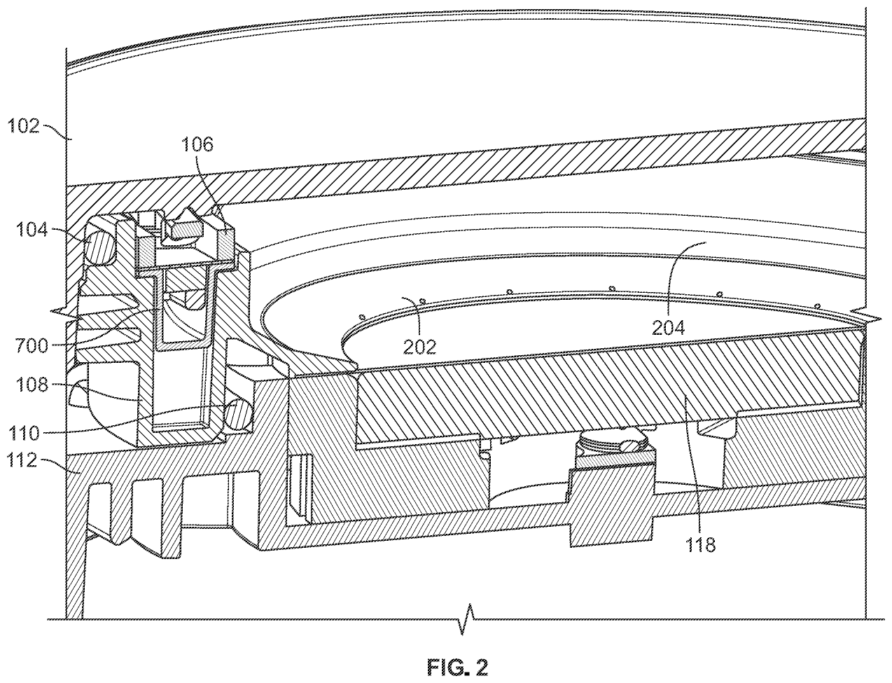

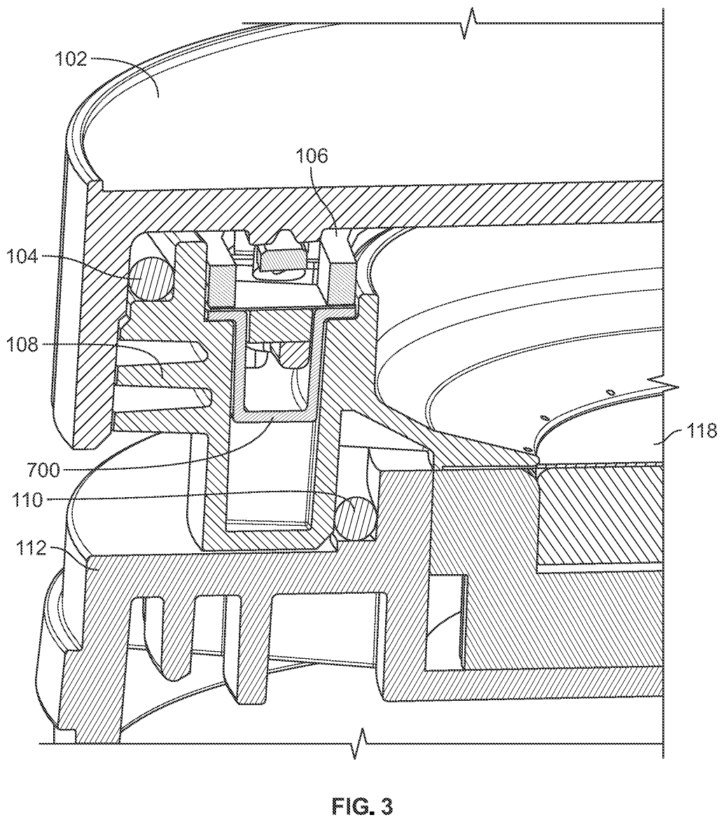

An example of a cassette assembly 100 is shown in FIG. 1, and a cross-sectional side-view is shown in FIG. 2 and FIG. 3. The cassette assembly 100 may provide a sterile environment for testing. The cassette assembly 100 may provide an anaerobic environment or an environment with a limited amount of oxygen in some embodiments.

From top to bottom in FIG. 1, the exemplary cassette assembly 100 includes a lid 102, an o-ring 104, an optional foil cutter 106, a tray assembly 700, a mid-body assembly 108, a membrane filter 118, a second o-ring 110, and a base assembly 112.

The base assembly 112 forms the bottom-most part of the cassette assembly 100 and serves as a supporting structure to which the other parts can be mounted. The base assembly 112 may be sized and shaped so as to be accommodated in an appropriate testing or analysis device.

A membrane filter 118 may be provided on the base assembly, between the base assembly 112 and the mid-body assembly 108. The membrane filter 118 may be a part of a media pad sized and shaped to be accommodated by a corresponding recess in the base assembly 112. The membrane filter 118 may be any suitable filter and may have characteristics (such as a desired porosity) selected based on the particular application (e.g., the size of the microorganisms of interest that are intended to be captured by the membrane filter 118). In some embodiments, more than one membrane filter 118 may be provided, which may include multiple different types of membrane filters 118.

Target fluids for analysis may be passed through the membrane filter 118 and into the base assembly 112. The base assembly 112 may include a drain port 116 that allows the fluids to be removed from the cassette assembly 100 after filtration. The drain port 116 may include an opening provided in a part of the base assembly 112 internal to the cassette assembly 100 that connects to a specially shaped outlet on the exterior side of the cassette assembly 100. The outlet may be sized and shaped to mate with a drain manifold that receives the removed fluid and delivers it to an appropriate disposal location.

An o-ring 110 may be provided between the base assembly 112 and the mid-body assembly 108 to prevent fluid from leaking around and therefore bypassing the membrane filter 118. The mid-body assembly 108 includes a mid-body inlet 114 that allows the target fluid (or fluids) being analyzed to be admitted into the cassette assembly 100. The mid-body inlet 114 may include an opening provided in a part of the mid-body assembly 108 internal to the cassette assembly 100 that connects to an opening on the exterior side of the cassette assembly 100. Within the mid-body inlet 114 may be a structure, such as a rubber septum, that seals the cassette assembly 100. To admit a target fluid into the cassette assembly 100, a needle may be used to pierce the structure in the mid-body inlet 114 and deliver the fluid at a relatively high pressure.

The top of the mid-body assembly 108 may be shaped to accommodate a tray assembly 700, which may include a scavenging material that (for example) absorbs oxygen in the cassette assembly 100 or other agents, such as described herein to create environmental conditions. More generally, the tray assembly may include desiccants, gases, or chemicals which react to create an environment to meet particular needs of organisms that are to be detected. For example, the tray may hold a desiccant to create a dry environment that is well suited for a target microbial. Similarly, the tray may hold a material for creating a sulfur rich environment that is desirable for certain microbes. In some embodiments multiple tray assemblies may be deployed to create desired environmental conditions. For instance, one tray may hold a desiccant and another may hold an agent to create a dry and sulfur rich environment.

The top of the mid-body assembly 108 may be shaped to accommodate a tray assembly 700, which may include a scavenging material that (for example) absorbs oxygen in the cassette assembly 100 or other agents, such as described herein to create environmental conditions. More generally, the tray assembly may include desiccants, gases, or chemicals which react to create an environment to meet particular needs of organisms that are to be detected. In some embodiments multiple tray assemblies may be deployed to create desired environmental conditions. In some embodiments, the tray assembly 700 may be topped by foil that holds the scavenging material in place and protects it from outside air until the tray assembly 700 is deployed in the cassette assembly 100. In other embodiments, a gas permeable material sock may be used rather than a gas impermeable foil. Such a gas permeable sock may be used when an environmental conditioning material is used in some embodiments. To release the scavenging material or the environmental conditioning material, the cassette assembly 100 may be provided with a foil cutter 106 designed to penetrate the foil or gas permeable sock and allow the scavenging material to scavenge the environment within the sealed cassette assembly 100 or allow the environmental conditioning material to condition the environment.

The use of the gas permeable barrier may eliminate the need for heat activated adhesives and allow the use of ultrasonic or heat welding of the tray seal. The gas permeable barrier also allows the use of environmental conditioning material that would otherwise react with a metal foil seal. The gas permeable barrier may be transparent or translucent to enable viewing of item in the tray, such as an indicator of seal integrity. The gas permeable barrier is not limited to filling of the tray by way of fill ports as with the foil seal. The gas permeable barrier may be integrated into a shelf on the tray and thus may reduce the number of parts needed.

To seal the cassette assembly 100, an o-ring 104 may be placed on top of the mid-body assembly 108, and then a lid 102 may be used to cap the entire assembly. As shown in FIG. 2 and FIG. 3, the o-ring 104 forms a seal between the mid-body assembly 108 and the lid 102 and prevents the fluid from leaking from the top of the cassette assembly 100 (and seals the interior of the cassette assembly 100 to allow the material to condition the environment).

As further shown in FIG. 2 and FIG. 3, the mid-body assembly 108 may include a mid-body assembly floor 202 that extends from an inner circumferential wall 204 of the mid-body assembly 108 towards an interior of the cassette assembly 100 in the radial direction. The mid-body assembly floor 202 may be slanted towards the membrane filter 118 to encourage the fluid to flow towards the membrane filter 118.

Although exemplary embodiments are described with reference to the depicted cassette assembly configuration for purposes of illustration, one of skill in the art will recognize that other types of cassette assemblies (with more, fewer, or a different configuration of parts) or other sterile environments may also be used.

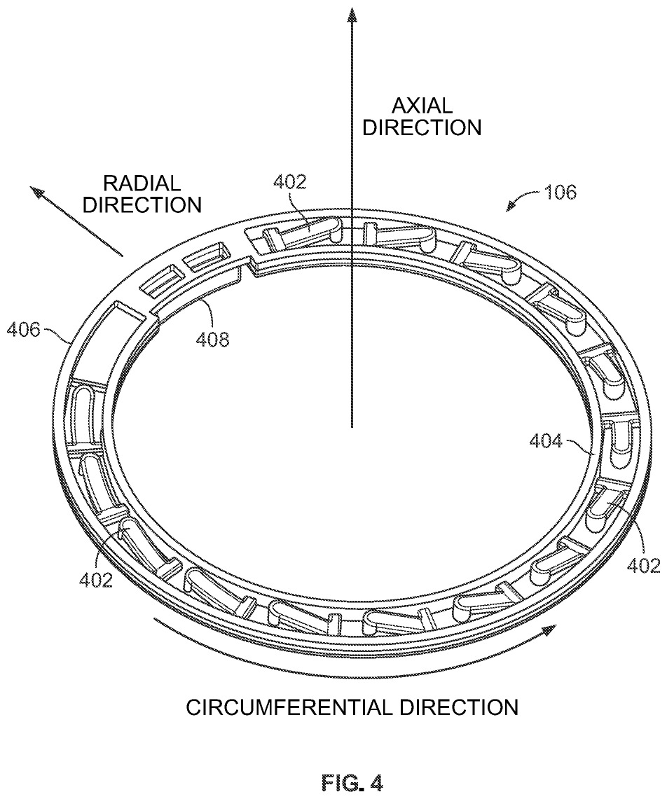

FIG. 4 is a perspective view of a foil cutter 106 in accordance with one embodiment. Note that, although this element is referred to as a foil cutter for ease of discussion herein, the tray is not limited to being sealed with foil. If another sealing material is used, a cutter configured to breach that type of sealing material may be constructed according to the principles described below.

As shown, the foil cutter 106 takes the form of a ring having a substantially planar surface 404 through which one or more openings are provided. Attached to the planar surface 404 and extending away from the planar surface 404 in an axial direction are one or more cutter teeth 402. The cutter teeth 402 may be non-rigid so that, when they are exposed to a force, they are capable of flexing and thereby extending into the openings. In use, the action of the lid may cause the cutter teeth 402 to come into contact with one or more protrusions, which force the cutter teeth down and into the foil seal of the tray assembly (this action is discussed in more detail below).

The foil cutter 106 includes at least one cutter tooth 402, but preferably includes multiple spaced around the circumference of the foil cutter 106. In this way, the foil cutter 106 can breach the foil seal of the tray assembly in several locations at the same time, resulting in an even exposure of the scavenging material throughout the environment.

Nonetheless, the cutter teeth 402 need not be provided around the entirety of the circumference; for example, FIG. 4 depicts a foil cutter 106 with a section 406 that is free of cutter teeth 402. This section may serve as a mounting point for tools or other features that extend out over the testing area (e.g., into the area above the mid-body assembly floor 202).

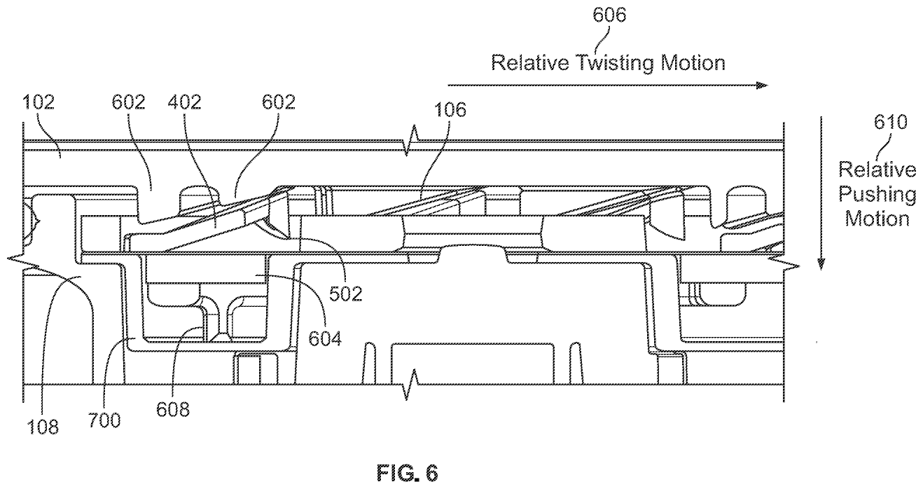

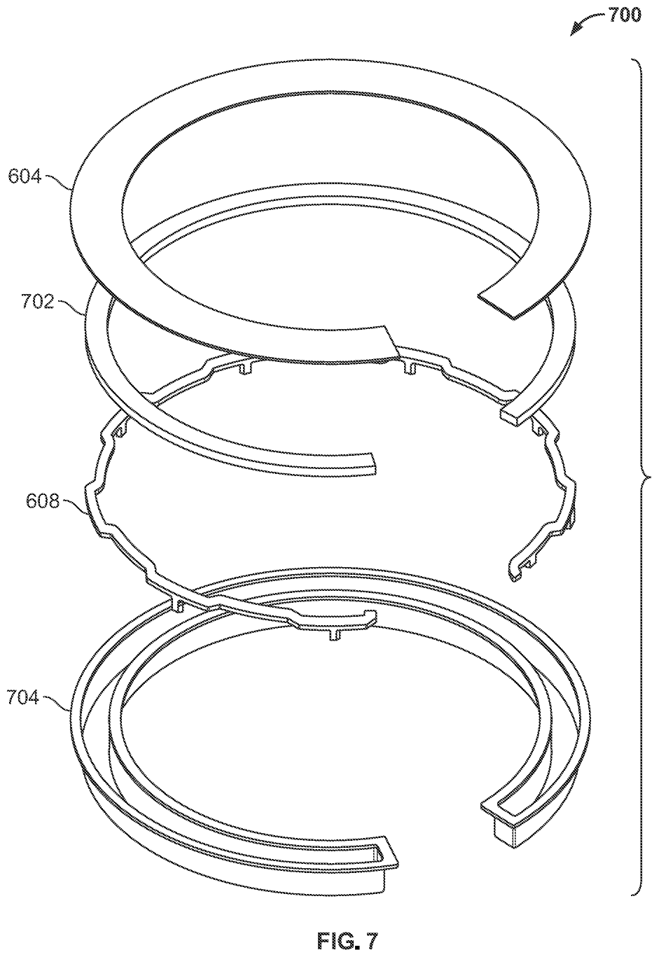

In some embodiments, the section 406 that is free of cutter teeth may include one or more alignment protrusions 408. As shown in FIG. 7, the tray may not be entirely ring shaped, instead having a cut-out section that results in the tray taking on the shape of a “C.” The alignment protrusion 408 may be aligned to the cut-out portion of the tray, and the teeth 402 may be configured so that when the foil cutter 106 is aligned via the alignment protrusion 408 in this way, the ramps 602 (see FIG. 6) are placed directly over the teeth 402 (in a push-to-engage configuration) or beside the teeth 402 (so that when the lid is rotated, the ramps ramp 602 engage with the teeth 402).

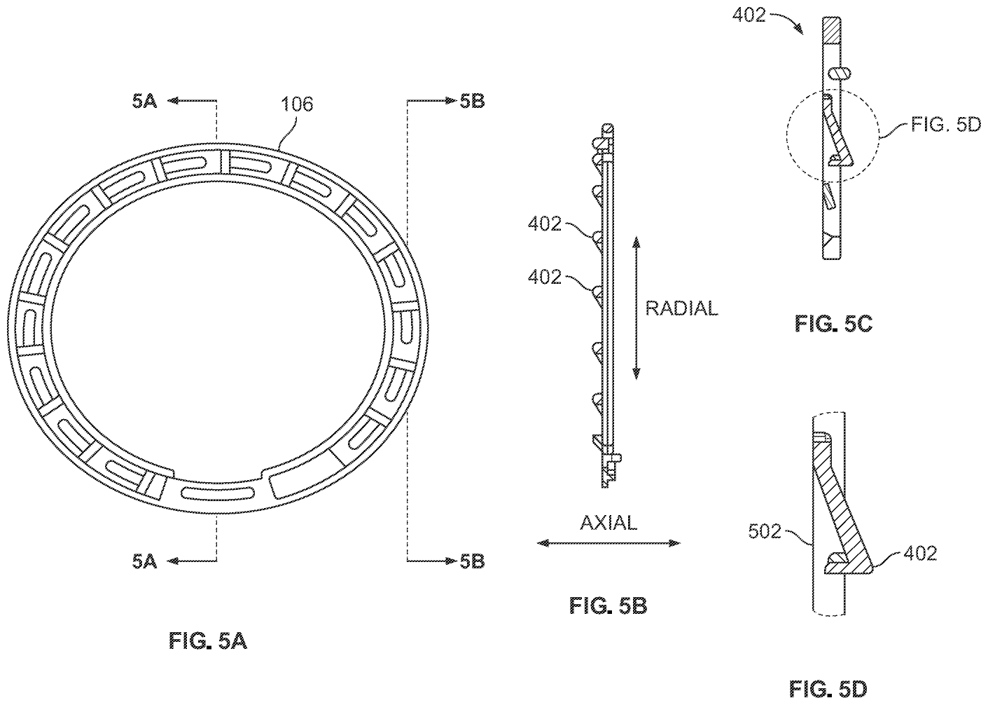

FIG. 5A is a top view of a foil cutter 106 in accordance with one embodiment. FIGS. 5B-5D depict various details of the foil cutter 106.

For example, FIG. 5B (depicting a side view of the foil cutter 106 taken along the line A-A in FIG. 5A) shows the cutter teeth 402. As illustrated, the teeth 402 extend away from the main planar surface of the foil cutter 106 in the axial direction. For reference and ease of discussion, in FIG. 5B, the left side of the diagram is considered to be axially upward, and the planar surface facing the left side of the image is considered to be the top of the foil cutter 106. The planar surface on the right side of the image is considered to be the bottom and faces an axially downward direction.

As more clearly seen in FIG. 5C and FIG. 5D, the teeth 402 of the foil cutter 106 extend into the openings in the planar surface. The axially top side of the teeth 402 may configured to mate with a ramp surface on the lid (see FIG. 6). This surface may be smooth so as to provide less resistance in a twist-to-activate embodiment; alternatively, this surface may be provided with one or more protrusions or tactile elements that provide feedback to the user as the teeth 402 engage with the ramps and flex. For example, the teeth 402 can be configured to resist or click when engaged with the ramps so that the user is informed that the teeth have engaged and penetrated the seal.

The axially bottom sides of the teeth 402 may terminate at a cutting tip 502 that is configured to penetrate the seal of the tray assembly when the ramp surface pushes down on the top of the teeth 402. This causes the cutting tip 502 to extend down through the opening and past the axially bottom portion of the foil cutter 106 (i.e., the bottom side of the planar surface), below which the seal of the tray may be positioned.

FIG. 6 is a cutaway side view showing the cutting action of the foil cutter 106 in accordance with exemplary embodiments.

The tray assembly 700 fits into a corresponding recess in the mid-body assembly 108 and is topped with a foil seal 604. The foil cutter 106 sits on top of the mid-body assembly 108 and tray assembly 700. When the teeth 402 are not engaged (as in the depiction in FIG. 6), the cutting tip 502 does not extend beyond the bottom surface of the foil cutter 106.

The lid 102 is placed on top of the mid-body assembly 108 and foil cutter 106. The lid 102 includes one or more ramps 602 configured to engage with the teeth 402. The size and/or shape of the teeth 402 may be selected in conjunction with the configuration of the ramp 602 so that, when the teeth 402 are engaged by the ramp 602, the teeth 402 extend a sufficient distance below the bottom surface of the foil cutter 106 so that the cutting tip 502 can make contact with an sufficiently penetrate the seal of the tray assembly 700 when it is deployed in the mid-body assembly 108.

The teeth 402 can be made to penetrate the foil seal 604 in several ways. For example, in one embodiment a user may twist the lid 102 with respect to the mid-body assembly 108 and base assembly 112, which may be locked together and move as a unit. The foil cutter 106 may move with the mid-body assembly 108/base assembly 112. When the lid 102 is twisted, it results in a relative twisting motion 606 that rotates the ramps 602 over the teeth 402 (or vice versa). The shape of the ramps 602 cause the teeth 402 to be pushed down and to flex, which pushes the cutting tip 502 into the foil seal 604 and penetrates it (thus exposing the scavenging material contained in the 700).

In another embodiment, the lid 102 may be aligned to the foil cutter 106 (e.g., by placing the lid 102 on the cassette using alignment features and/or by slightly rotating the lid 102 until alignment features are engaged to indicate that the ramps 602 are aligned to the teeth 402) and then pushed to engage the ramps 602 with the teeth 402. In this embodiment, there is a relative pushing motion 610 between the lid 102 and mid-body assembly 108/base assembly 112, which may move with the foil cutter 106. The pushing motion may cause the ramps 602, which may be aligned with the teeth 402, to cause the teeth 402 to flex and push the cutting tip 502 into the foil seal 604.

In other embodiments, the material in the tray may be activated in other manners. For example, the materials may be activated magnetically. Magnetic elements may apply a magnetic field that either causes actuation of elements or acts upon the material to cause activation of the material. Further, the material may be activated by environmental conditions. like temperature or humidity. For example, a wax motor or another mechanism may be used to convert temperature into mechanical actuation to bring about the activation. Pressure may also be used to realize actuation of a seal or activation of the material. Still further, a push button activation mechanism may be used to activate the material.

FIG. 7 is an exploded view of an exemplary tray assembly 700 in accordance with one embodiment.

The tray assembly 700 may include an tray 704 into which the material may be added. The tray 704 may be sized and shaped to fit into a corresponding recess in the mid-body assembly 108. The tray 704 may be annular or substantially annular (e.g., “C”-shaped) and may be sized and shaped to at least partially correspond to the size and shape of the foil cutter 106. By providing an annular tray 704, the material can be provided around most or all of the perimeter of the area in which the sample will be tested, which allows for an even distribution when the foil seal 604 is pierced by the foil cutter 106.

An axially top side of the tray assembly 700 is sealed by a foil seal 604 (although other types of sealing materials other than foil may also be used). The foil seal 604 may be annular and sized and shaped to fit over the top of the tray 704. The edge(s) of the foil seal 604 may be coated in an adhesive material (such as a heat-activated adhesive material) to allow the foil seal 604 to be affixed to the tray 704 and create an air-tight seal on the upper surface.

The axially bottom side of the tray 704 includes one or more fill ports (not visible in FIG. 7; see FIG. 9). The material may be added to the tray 704 through the fill ports, and then the fill ports may be sealed in an air-tight manner.

When the foil seal 604 is pierced during use, it may be preferable that the scavenging material does not exit the tray 704 and contaminate the sample being tested. Accordingly, exemplary embodiments provide a shelf 608, which may be a molded shelf (e.g., formed of plastic or another suitable material). The shelf 608 may accommodate a foam insert 702 that sits on the shelf 608 and is sized and shaped to prevent the scavenging material (accommodated in the bottom part of the tray 704) from exiting through the penetrations in the foil seal 604.

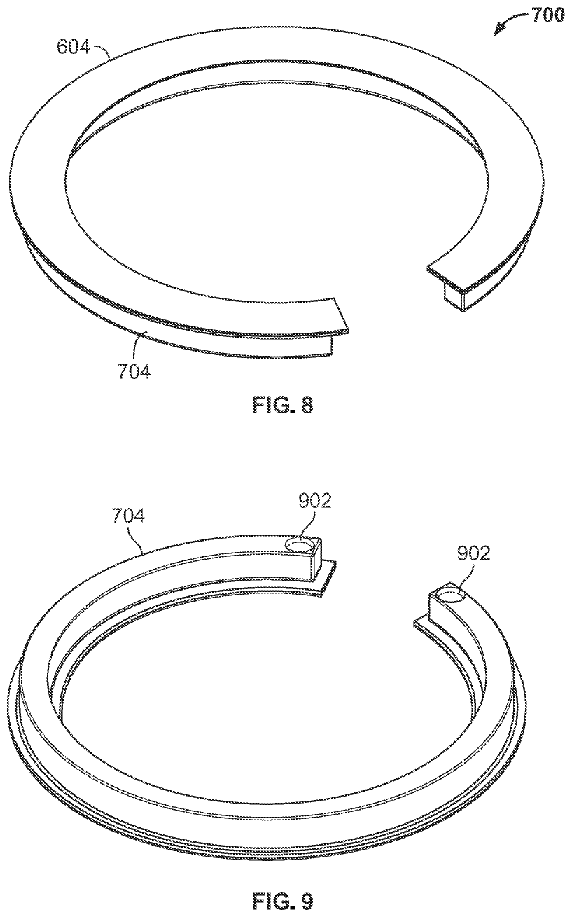

FIG. 8 is a perspective view depicting the assembled tray assembly 700 from the top in accordance with one embodiment.

Meanwhile, FIG. 9 shows the assembled tray assembly 700 from the bottom in accordance with one embodiment. FIG. 9 shows the fill ports 902 provided on the bottom of the anaerobic tray 704, though which the material may be introduced to the tray 704.

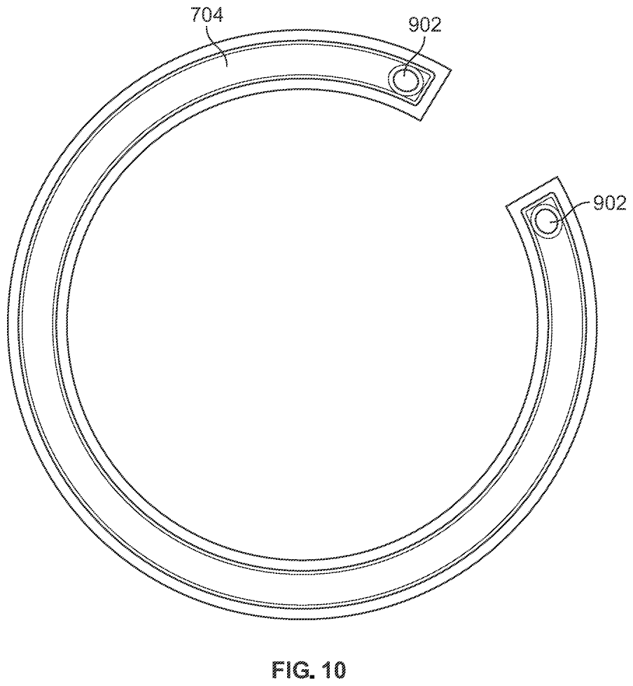

The fill ports fill ports 902 are also visible in FIG. 10, which depicts a further bottom view of an assembled tray assembly 700.

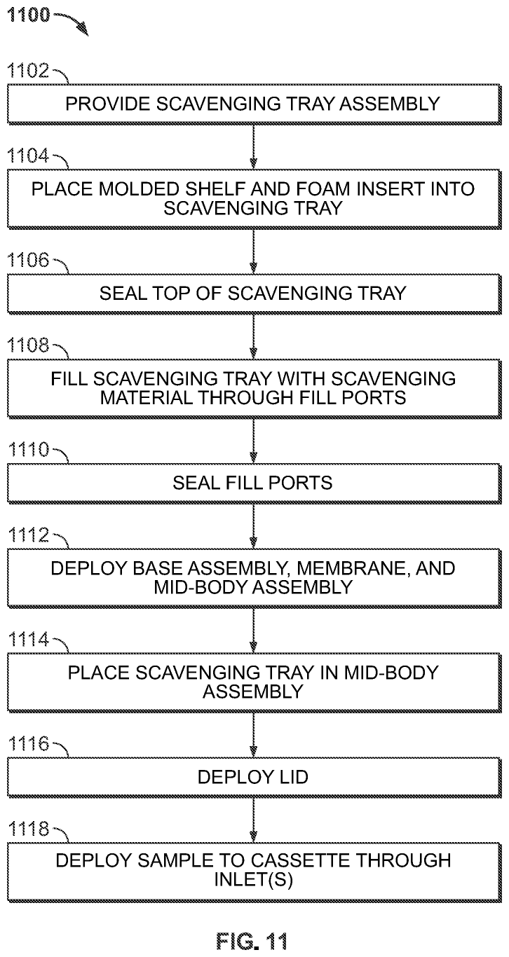

FIG. 11 is a flowchart depicting an exemplary method for creating an environment in a cassette assembly in accordance with one embodiment.

In block 1102, a user may provide a tray assembly including at least an anaerobic tray and a sealing element (such as a foil ring coated at the edges with an adhesive).

In block 1104, the user may optionally assemble the tray by placing an optional molded shelf and foam insert into the tray. As noted above, these components may be provided to prevent the oxygen-scavenging material from leaking into the testing environment when the sealing element is pierced.

In block 1106, the top of the tray may be sealed. For example, if the sealing element is a foil ring coated with a heat-sensitive adhesive, the foil ring may be placed on top of the anaerobic tray and heat may be applied to activate the adhesive. The user may optionally test the seal to ensure that it is air-tight.

In block 1108, the user may turn the tray over to expose the bottom fill ports and may fill the tray with the material through the fill ports. As the tray is filled, the tray may be manipulated (e.g., turned, tapped, etc.) to ensure that the material is distributed throughout the tray.

After a predetermined amount of scavenging material is added to the tray, in block 1110 the fill ports may be sealed. The fill ports may be sealed with foil in a similar manner to the foil seal on top of the tray, or using another type of seal (e.g., a plug). The user may test the seal over the fill ports to ensure that it is airtight.

In block 1112, a base assembly, membrane, and mid-body assembly may be deployed. For example, the base assembly may be placed on a suitable surface in a testing area, and the membrane (and/or a media pad) may be placed on the base assembly. An o-ring may be put into position around a circumference of an appropriate portion of the base assembly. A mid body assembly may then be lowered onto the base assembly, so that the o-ring creates a seal and/or locks the mid-body assembly to the base assembly.

In block 1114, the user may place the filled tray assembly in a corresponding recess in the mid-body assembly. The foil cutter 106 may be placed on top of the tray.

In block 1116, the lid may be deployed on the cassette assembly. This may involve providing an o-ring at a suitable location on the mid-body assembly, and then securing the lid at an inner or outer circumference of the o-ring.

The lid may initially be partially secured, such that the foil cutter 106 does not engage with the ramps 602 to penetrate the foil seal 604. The cassette assembly may be moved to the location where it will be filled or tested, and then the lid may be fully secured. Depending on the mechanism of action of the foil cutter 106, the lid may either be pushed to close, or may be twisted. In either event, closing the lid may cause the ramps 602 to engage with the teeth 402, which pushes the cutting tip 502 into the foil seal 604. The cutting tip 502 penetrates the foil seal 604 and exposes the material to the now-sealed internal environment of the cassette.

In block 1118, a fluid delivery device may be inserted into an inlet of the mid-body assembly. For example, the inlet may include a rubber septum. The rubber septum may be pierced with a needle, and the fluid may be delivered to the inlet through the needle. The fluid may be delivered through one or more tubes attached to the needle, or through another suitable delivery device.

The fluid may be passed through the membrane. The fluid may then be released through an outlet in the base assembly. After the fluid is released, a growth medium may be provided to the vicinity of the membrane (e.g., through the mid-body inlet or another suitable inlet), and the cassette assembly may be left to incubate for a predetermined period of time. Any growth of microorganisms on the growth medium may then be measured (e.g., by imaging the growth medium through the lid, which may be optically transparent).

In some embodiments a hybrid design may be used in which polymer may be used for support arms of the cutter teeth. One of the advantages of using the polymer support arms is that the polymer support arms enables the cutter head to spring back after cutting. This may simplify the design of the foil cutter 106 and avoid accidental cutting.

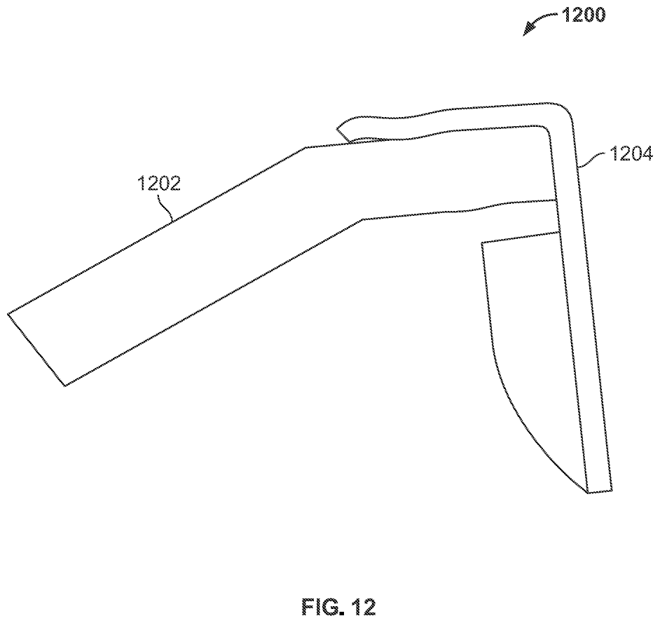

FIG. 12 shows an example portion of a cutter tooth arrangement 1200 with a polymer support arm. The cutter head 1204 is formed of a suitable metal but the support arm 1202 is formed of a polymer, such as a plastic. The use of the metal cutter head 1204 is beneficial in that the metal cutting head can be formed to create a larger hole than if the cutter tooth assembly were wholly formed of a polymer. The resulting larger hole may allow for more efficient scavenging. Wholly plastic cutters generally cannot puncture or cut gas permeable films, whereas the metal cutter head can cut through gas permeable membranes. As can be seen, the cutter head 1204 is shaped like an arrowhead. The combination of the support arm 1202 with the metal cutter head 1204 is more cost effective to produce than an all metal cutter tooth arrangement.

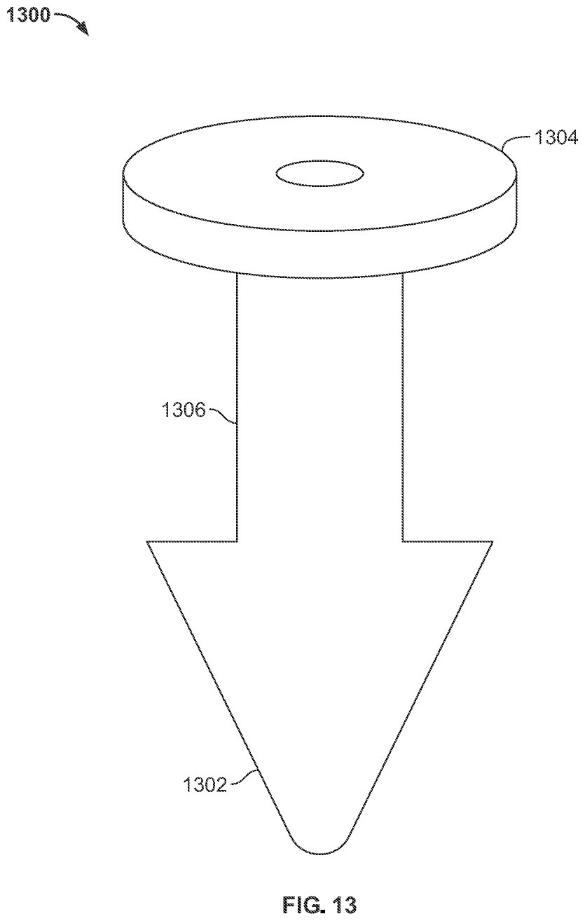

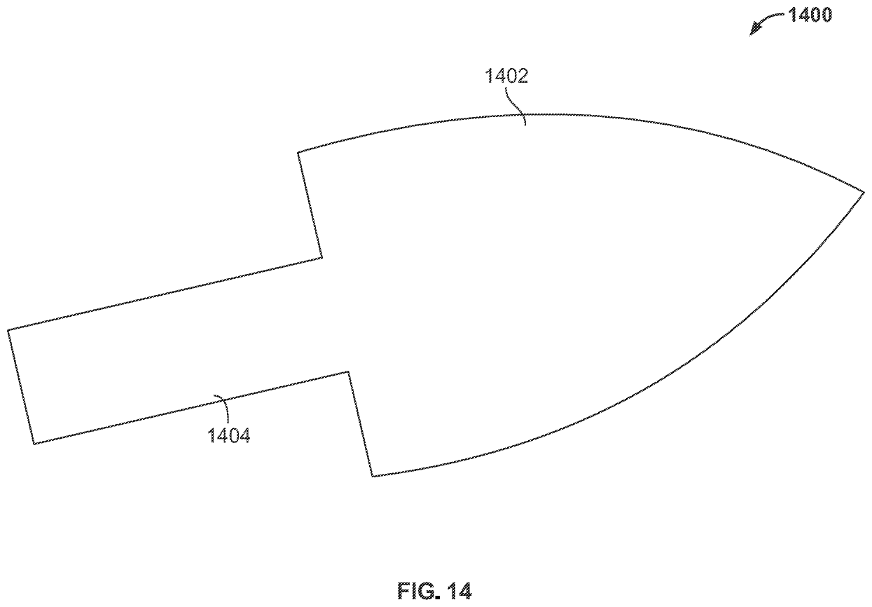

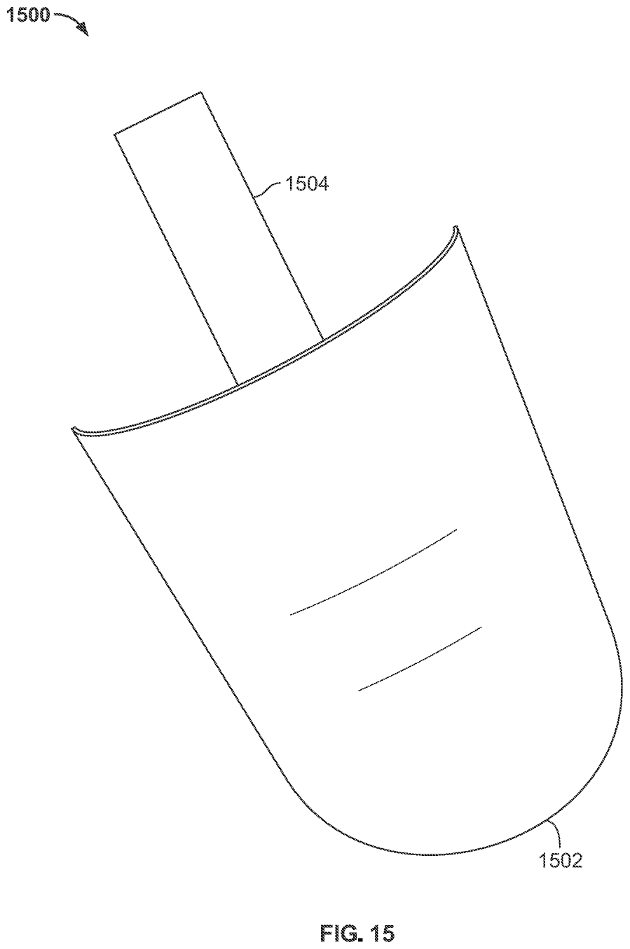

FIG. 13 shows the cutter head 1300 from a front view. The cutter head includes the portion 1304 that is connected to the support arm, a neck portion 1306, and the arrowhead portion 1302 for tearing the foil or other sealing layer in the cassette assembly. FIG. 14 shows the cutter head 1400 with a flat profile. This view includes the arrowhead portion 1402 and the neck portion 1404. FIG. 15 shows an alternative arrangement of the cutter head 1500 where the leading portion 1502 has a curved profile rather than a flat profile. Neck portion 1504 is also shown.

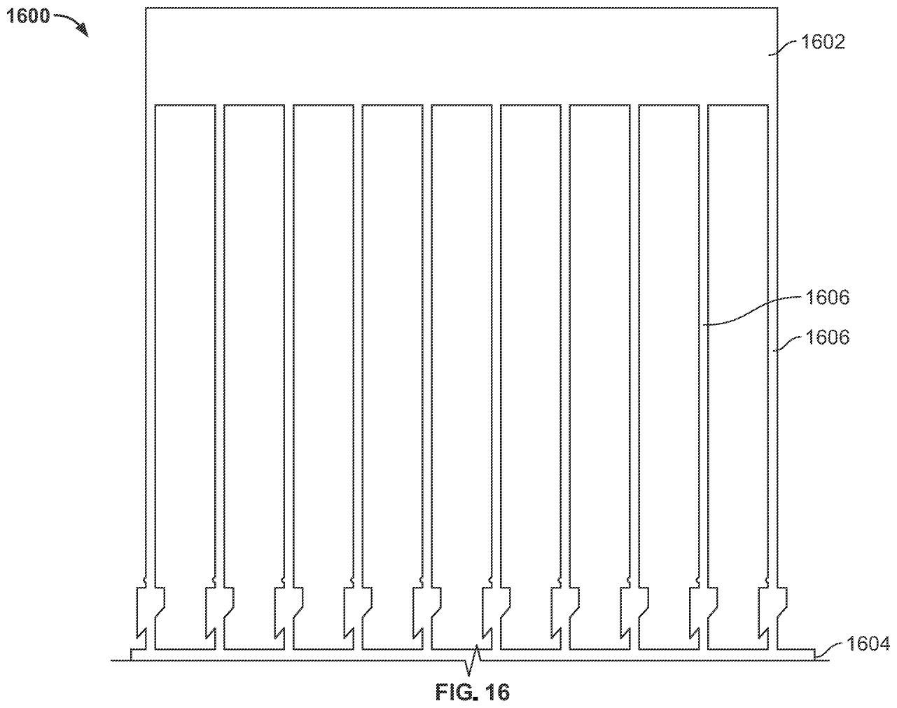

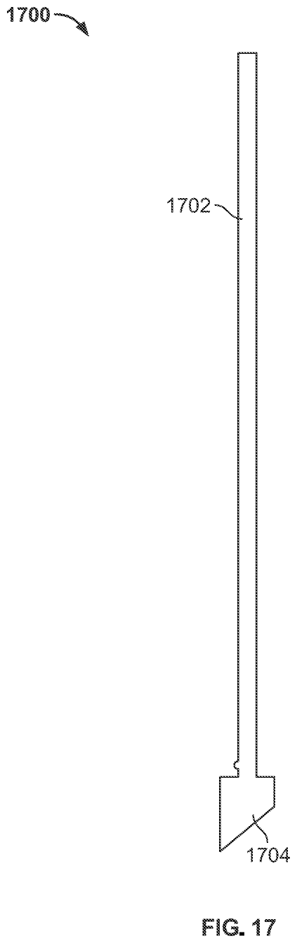

FIG. 16 shows an example of how the metal cutter head can be formed by die cutting, punching or photo-etching. As shown a metal sheet 1600 has ben processed to create several elements 1606 that are connected to a top portion 1602 and a metal portion 1604 (only partially shown). As shown in FIG. 17, an element 1700 includes a projection 1702.

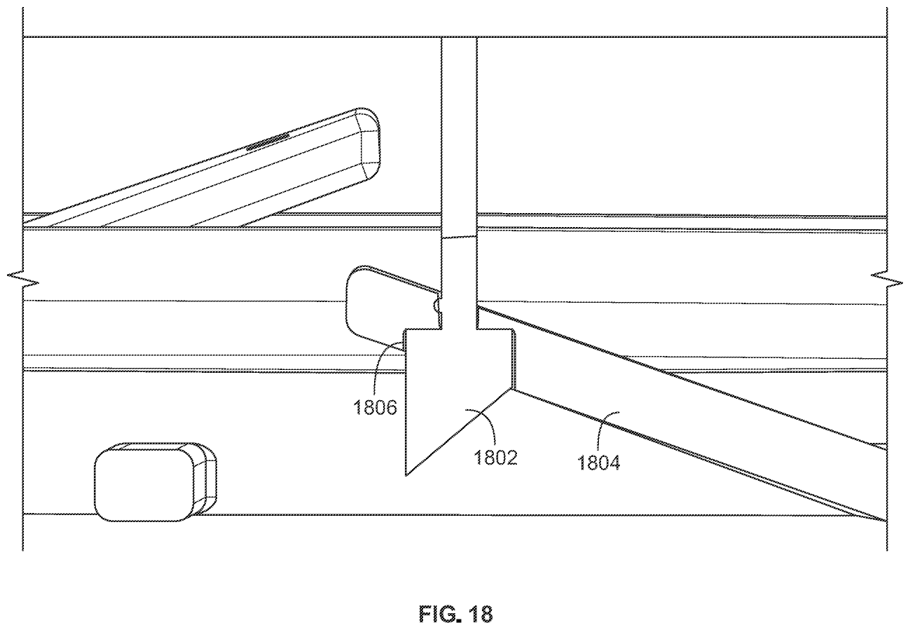

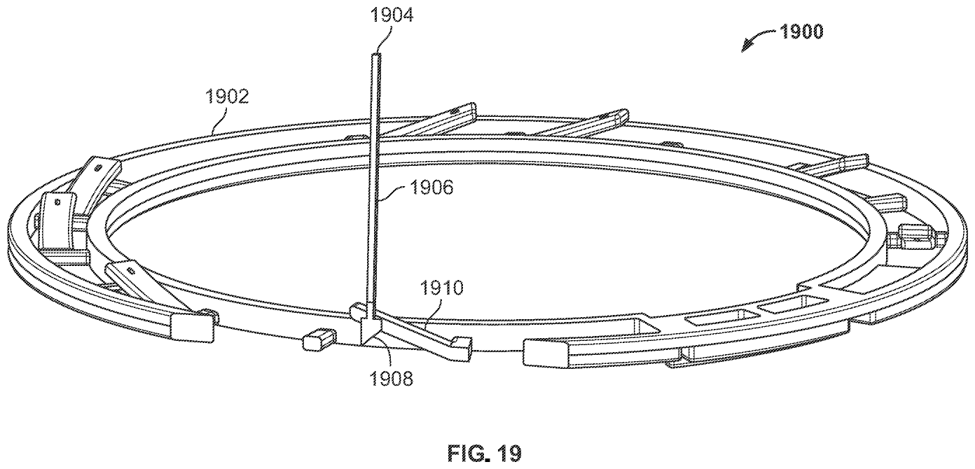

FIG. 18 shows how such an element may be fit into the foil cutter 106, such as described above. The cutting edge 1802 is inserted and installed a recess 1806 of a ramp 1804. The cutting edge 1802 may be configured to have a frictional fit with the recess 1806 or alternative holding mean may be provided to securely connect the cutting edge 1802 in the recess 1806. FIG. 19 depicts an example foil cutter 1900 with a ring structure 1902. The element 1904 is installed. The projection 1906 is still in place, and the cutting edge 1908 is positioned in the recess of the ramp. Once the cutting edge 1908 is securely installed, the projection 1906 may be removed, such as by trimming the excess off.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

With general reference to notations and nomenclature used herein, the detailed descriptions herein may be presented in terms of program procedures executed on a computer or network of computers. These procedural descriptions and representations are used by those skilled in the art to most effectively convey the substance of their work to others skilled in the art.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

1. A cutter for a cassette assembly comprising a tray topped with a seal and a lid, the cutter comprising:
an annular planar surface comprising one or more openings;
at least one flexible cutter tooth extending away from an axial top of the planar surface, the flexible cutter tooth configured to interact with the lid in order to cause the flexible cutter tooth to flex; and
a cutting tip on each of the at least one flexible cutter teeth, wherein, the at least one flexible cutter tooth is sized so that, when engaged with the lid, the cutting tip penetrates the seal on the tray.

2. The cutter of claim 1, wherein the at least one cutter tooth is sized so that, when not engaged with the lid, the cutting tip is disposed in one of the openings and does not extend beyond an axial bottom of the cutter.

3. The cutter of claim 1, wherein the at least one cutter tooth is configured to interface with a ramp on the lid of the cassette assembly to force the cutting tip into the seal

4. The cutter of claim 1, wherein the cutter is a ring separate from the lid.

5. The cutter of claim 1, wherein the sealing material is a foil or a gas-permeable membrane.

6. The cutter of claim 1, wherein the one or more flexible cutter teeth comprise a plurality of teeth arranged substantially around the circumference of the cutter.

7. The cutter of claim 6, further comprising a tooth-free region on the cutter configured to receive a tool.

8. The cutter of claim 1, wherein the cutter is configured to penetrate the foil upon a relative twisting motion between the lid and a remainder of the cassette assembly.

9. The cutter of claim 1, wherein the cutter is configured to penetrate the foil upon a relative pushing motion between the lid and a remainder of the cassette assembly.

10. The cutter of claim 1, wherein the cutter is sized and shaped to surround an internal testing environment of the cassette assembly.

11. A method comprising:
assembling the cutter of claim 1 into the cassette assembly; and
seating the lid onto the cassette assembly to cause the cutting tip to penetrate the seal.

12. The method of claim 11, wherein seating the lid comprises pushing the lid onto a remainder of the cassette assembly.

13. The method of claim 12, further comprising aligning one or more ramps on the lid with the at least one flexible cutter teeth of the cutter.

14. The method of claim 11, wherein seating the lid comprises twisting the lid over a remainder of the cassette assembly.

15. The method of claim 14, wherein twisting the lid rotates one or more ramps on the lid into the at least one flexible cutter teeth of the cutter.

16. The method of claim 11, further comprising placing the tray into a recess in the cassette assembly.

17. The method of claim 16, wherein assembling the cutter into the cassette assembly comprises placing the cutter on top of the tray.

18. The method of claim 11, wherein seating the lid onto the cassette assembly creates an air-tight seal.

19. The method of claim 11, further comprising deploying a tool on a tooth-free region on the cutter.

20. The method of claim 11, further comprising aligning a protrusion on an axial bottom of the cutter with a cut-out portion of the tray.

21-40. (canceled)
